(19) United States
(12) Patent Application Publication
(10) Pub. No.: US 2024/0424258 A1
(43) Pub. Date: Dec. 26, 2024

(54) CATHETERIZATION DEVICE

(71) Applicant: VascoMed GmbH, Binzen (DE)

(72) Inventors: Ingmar Christiaan MAURICE, Amsterdam (NL); Johannes HARTWIGER, Freiburg (DE)

(73) Assignee: VascoMed GmbH, Binzen (DE)

(21) Appl. No.: 18/706,031

(22) PCT Filed: Oct. 20, 2022
(86) PCT No.: PCT/EP2022/079234

§ 371 Date: Apr. 30, 2024

(30) Foreign Application Priority Data
Nov. 1, 2021 (EP) 21205753.3

(51) Int. Cl. A61M 25/01

(52) U.S. Cl. CPC A61M 25/0136

(57) ABSTRACT

Catheterization device comprising a catheter including a deflectable steering end having a rest position. In order to provide a sensitive control of the steering end of the catheterization device that avoids sudden jumping movement of the steering end at the rest position the catheterization device comprises a control device for controlling the deflectable steering end, wherein the control device comprises a control arrangement having an elongated element which is mounted rotatably about a first pivot axis, wherein the control arrangement further comprises at least one guide element arranged at a distance from the first pivot axis, wherein at least one the guide element is rotatable with the elongated element about the first pivot axis or the guide element is rotatable mounted and coupled to the rotational movement of the elongated element, wherein the at least one guide element is connected in a movement-transmitting manner to the steering end.

### CROSS-REFERENCE TO RELATED APPLICATIONS

This application is the United States National Phase under 35 U.S.C. § 371 of PCT International Patent Application No. PCT/EP2022/079234, filed on Oct. 20, 2022, which claims the benefit of European Patent Application No. 21205753.3, filed on Nov. 1, 2021, the disclosures of which are hereby incorporated by reference herein in their entireties.

### TECHNICAL FIELD

The present invention is generally directed to a catheterization device, which has a catheter including a deflectable steering end having a rest position, and a control device for controlling the deflectable steering end, wherein the control device comprises a control arrangement having an elongated element which is mounted rotatably about a first pivot axis and which has at least one guide element arranged at a distance from the first pivot axis and rotatable with the elongated element about the first pivot axis, wherein the at least one guide element is connected in a movement-transmitting manner to the steering end.

### BACKGROUND

Catheterization devices of the type mentioned above are known in general and are used for the examination and treatment of patients, for example, suffering from heart conditions. In order to guide the catheter to the heart, for example, and to examine and/or treat a selected region of the heart, a distal end of the catheter is formed as the steering end. The steering end can be controlled and, in particular, bent into at least one, two, or more than two different directions by a proximal end of the catheter arranged opposite the distal end along the catheter. To bend the steering end, the steering wire, for example, is guided from the steering end to the proximal end, where it is fixed to the control arrangement. If the control device is operated such that the elongated element is rotated about the first pivot axis, it pulls on the steering wire and a force attempting to deflect the steering end is conveyed to the steering end via the steering wire.

Document European Patent No. 2 853 284 B1 describes a catheterization device of the above type which comprises an energy store connected to the control arrangement via a force transmission element. The energy store produces an auxiliary force assisting the rotation of the control arrangement aiming at easy operation at low application of force for the operator.

At and around the rest position of the deflectable steering end an operation of the control device may result in unwanted sudden, e.g., jumping movement of the steering end caused by necessary slack in the steering wire and torque on the elongated element provided by the energy store if the pivot angle of the control arrangement is not exactly at its zero position. The torque produced by the energy store forms an instable force compensation mechanism that compensates for the opposing force of the catheter.

The present disclosure is directed toward overcoming one or more of the above-mentioned problems, though not necessarily limited to embodiments that do.

### SUMMARY

Accordingly, an objective to be solved is to provide a sensitive control of the steering end of the catheterization device that avoids sudden jumping movement of the steering end.

At least the above objective is solved by a catheterization device with features of claim 1.

In particular, the above problem is solved by a catheterization device of the type described above, wherein the control device comprises a control arrangement having an elongated element which is mounted rotatably about a first pivot axis, wherein the at least one guide element is rotatable with the elongated element about the first pivot axis or the at least one guide element is rotatable mounted and coupled to the movement of the elongated element, wherein the control arrangement further comprises an energy storage element, which produces an auxiliary force assisting the rotation of the elongated element, wherein the elongated element has a force transmission element which is arranged at a distance from the first pivot axis and from the at least one guide element and which is coupled in a force-transmitting manner to a first end of the energy storage element, wherein the energy storage element comprises a first rolling surface located at a second end of the energy storage element opposite to the first end and extending substantially parallel to the first pivot axis, wherein the first rolling surface is configured such that, if the elongated element rotates about the first pivot axis within a pre-defined angular range corresponding to the rest position of the steering end, the first rolling surface rolls along a second rolling surface thereby executing a combined translational and rotational movement along the second rolling surface between a second pivot axis and a third pivot axis, wherein the second pivot axis and the third pivot axis extend substantially parallel to the first pivot axis.

The deflectable steering end is formed by the distal end of the catheter and may be inserted into a body or alike and is steerable by the control device, which is the handheld part of the catheterization device located at the distal end, by which an operator controls the deflectable steering end.

In order to deflect the deflectable steering end from the rest position into a deflected position, the deflectable steering end is connected in a movement-transmitting manner to the guide element by means of the at least one steering wire. For example, a first end of the at least one steering wire is connected to the deflectable steering end and a second end of the steering wire is connected to the guide element of the control arrangement.

The rest position describes a position of the deflectable steering end at which the steering end is not deflected and substantially straight.

The control arrangement has an elongated element which is mounted rotatably about a first pivot axis. The elongated element is, for example, a stiff, rot-like element. In a first embodiment, the at least one guide element arranged distanced from the first pivot axis and is rotatable about the first pivot axis according to the rotation of the elongated element. In a second embodiment, the at least one guide element is not directly rotated by the elongated element about the first pivot axis but is rotatable mounted in a distance from the first pivot axis (i.e., the guide element pivot axis of the at least one guide element is different, e.g., parallel to the first pivot axis) and the rotation of the guide element about the guide element pivot axis is driven by the rotational movement of the elongated element or drives the rotation of the elongated element. Accordingly, a rotating movement of the elongated element about the first pivot axis results in a rotation of the guide element and thus in a deflection of the deflectable steering end. The elongated element may rotate about the first pivot axis into one direction or into two opposite directions, so that the steering end may be deflected into one direction or into two directions from the rest position.

The elongated element provides a rotational movement about the first pivot axis which corresponds to the operation of the control device by the operator, e.g., the Health Care Practitioner (HCP). The elongated element is connected to the at least one steering wire via the at least one guide element and to the compensating force providing energy storage element. In the rest position of the steering end the elongated element has a corresponding rest position. Due to a slack in the steering wire, the compensating force is not applied to the elongated element if the elongated element is rotated within a pre-defined angular range about this rest position by a respective operator manipulation of the control device in order to avoid sudden jumping movement of the steering end as explained in detail below.

The force transmission element and the first end of the energy storage element are connected in a force transmitting manner. Further, the at least one guide element is connected to the steering end and the energy storage element assists in rotation of the elongated element such that an opposing force from the deflected deflectable steering end may be compensated by the energy storage element. This allows, that the deflectable steering end remains in its set position until the operator sets it to a different position and hence deflecting state and that the force necessary for deflection and introduced manually into the control arrangement is reduced such that the burden on the operator of the catheterization device decreases and the catheterization device can be operated more easily. The energy storage element provides a compressive force acting into the direction of the force transmitting element and thereby into the direction of first pivot axis and the elongated element. If the compressive force does not exactly act into the direction of the first pivot axis, a torque is produced attempting to rotate the elongated element further away from its rest position.

The energy of the energy storage element provided to the force transmitting element may be a compressive force or a tensile force as indicated in detail in document European Patent No. 2 853 284 B1 which is herewith included by reference. The energy storage element further may be a pneumatic energy storage element or an elastic and may provide, in particular, a spring-elastic force to assist the operation of the control arrangement. For example, the energy storage element may comprise a tension spring or a compression spring, for example, a coil spring, which results in low costs.

The second end of the energy storage element comprises the first rolling surface, which interacts with the second rolling surface. The first and the second rolling surface extend basically parallel to the first pivot axis, wherein the movement of the first rolling surface along the second rolling surface is generally perpendicular to the first pivot axis. Accordingly, the first rolling surface and the second rolling surface extend additionally perpendicular to the first pivot axis. The second rolling surface may be formed by a plate-like element, in particular by a surface of the plate-like element opposite the first rolling surface. With regard to the movement of the first rolling surface along the second rolling surface two angular regions are distinguished, namely whether the rotation of the elongated element about the first pivot axis is within the pre-defined angular range corresponding to the rest position of the steering end or whether the rotation is outside of this pre-defined angular range. The first and second rolling surfaces are substantially planar or slightly curved, however, adapted in their form to each other in order to realize a “rocking-chair” movement as indicated below. If the elongated element rotates within the pre-defined angular range corresponding to the rest position of the steering end, the first rolling surface of the energy storage element at the second end of the energy storage element rolls along the second rolling surface in a combined translational and rotational movement between the second and the third pivot axis thereby forming the “rocking chair” movement. The translational movement is substantially perpendicular to the first pivot axis. This movement causes that the vector of the auxiliary force produced by the energy storage element is directed to the first pivot axis along the whole movement range between the second and the third pivot axis. Accordingly, no torque results from this movement thereby preventing jumping movements of the steering end. As soon as the elongated element pivots further and leaves the pre-defined angular range, the second end of the energy storage element pivots around the second pivot axis or third pivot axis thereby causing the vector of the auxiliary force pointing not into the direction of the first pivot axis, producing a torque and supporting the rotational movement of the at least one guide element leading to a substantial deflection of the steering end.

The first pivot axis, the second pivot axis and the third pivot axis are arranged substantially parallel to each other and are arranged distanced to each other.

The energy storage element pushes against the elongated element via the force transmission element with a force, resulting in a torque applied to the control arrangement, unless the force vector points along the extension of the elongated element into the direction of the first pivot axis. In the case where the force points directly in the direction of the first pivot axis, in the rest position or around the rest position, i.e., within the pre-defined angular range, no torque is applied by the energy storage element to the control arrangement.

In one embodiment, the second rolling surface comprises a first rib and a second rib extending substantially parallel to the first pivot axis, wherein the rolling movement of the first rolling surface along the first rib causes rotation of the second end of the energy storage element about the second pivot axis and wherein the rolling movement of the first rolling surface along the second rib causes rotation of the second end of the energy storage element about the third pivot axis if the elongated element rotates about the first pivot axis outside the pre-defined angular range. A cross section of first rib and/or the second rib form, for example, a segment of a circle. The second pivot axis substantially forms the axis of the second rib, while the third pivot axis substantially forms the axis of the third rib. In another embodiment, the form of the cross section may be adapted according to the operator's needs. The ribs create not only defined pivoting axes but also the end sections of the rocking chair movement of the first rolling surface and the second rolling surface. This means, that by the distance of the first rib and the second rib along the first rolling surface the size of the predefined angular range of the elongated element is determined.

In one embodiment, the first rolling surface comprises a first recess and a second recess, wherein the first recess is adapted to be engaged with the first rib and the second recess is adapted to be engaged with the second rib. Further, the rib engages with the respective recess when the elongated element rotates about the first pivot axis outside of the pre-defined angular range, creating defined second and third pivot axes. By the engagement of the rib with the respective recess, the second and/or third pivot axes are well defined, such that the operator can finely operate the deflectable steering end outside the pre-defined angular range defined above.

In one embodiment, the pre-defined angular range about which the elongated element rotates about the first pivot axis is an angular range between 100 and −10°, in particular between 7° and −7°, in particular between 4° and −4°, wherein the angular value 0° of the elongated element corresponds to the rest position of the steering end. If the elongated element rotates within this pre-defined angular range, the first rolling surface rolls along the second rolling surface between the second pivot axis and the third pivot axis and the auxiliary force produced by the energy storage element points into the direction of the first pivot axis. This angular range corresponds with the slack in the steering wire so that it makes sense to choose the pre-defined angular range as indicated above. Accordingly, the pre-defined angular range is adapted, for example, to the operator's preferences and/or to the slack of the at least one steering wire. If, for example, the slack is increased, the pre-defined angular range needs to be increased, as well, wherein the deflectable steering end is not deflected if the elongated element is rotated about the first pivot axis within this pre-defined angular range.

In one embodiment, the control device comprises a frame and a hull, which is displaceable relatively to the frame thereby causing rotation of the elongated element about the first pivot axis, wherein the displacement direction extends along a longitudinal hull axis, and wherein the first pivot axis and the second rolling surface are fixed to the frame. In case the second rolling surface is formed by a plate-like element, the plate-like element is fixed to the frame. The frame and the hull are adapted to the dimensions of an operator's hand and a possible displacement dimension. Alternatively, the displacement of the hull relative to the frame may be a rotational displacement or a combined displacement extending along the longitudinal hull axis and a rotation. The outer surface of the hull may be formed as a section of a cylinder or a cone. In one embodiment, the hull is a hollow element, wherein the frame is located within the inner lumen of the hull and may be adapted to the form of the inner lumen. In one embodiment, the distal section of the frame may extend over the distal end of the hull forming a head as a contact surface for at least one of the operator's fingers. This arrangement allows an easy assembly, too. Furthermore, the operator can hold the hull of the catheterization device in his hand, such that the operator can operate the catheterization device in an ergonomic manner using only one hand. This further allows a fatigue-free and well-defined operation.

In one embodiment, the elongated element is a toggle lever pivotable around the first pivot axis and the control arrangement further comprises a gear member, wherein a first end of the toggle lever engages with the gear member comprising the at least one guide element and a second end of the toggle lever is coupled to the force transmission element. The toggle lever comprises at least one tooth or similar connecting element which provides the engagement of the toggle lever with the gear member at its first (e.g., distal) end. The gear member transmits its rotational movement to the toggle lever and but is assisted by the torque produced by the energy storage element if the rotation angle of the toggle lever is outside the predefined angular range.

In one embodiment, the toggle lever transmits the torque provided by the energy storage element to a tension disc as the guide element arranged at the gear member to compensate the opposing force of the steering end of the catheter via the tension disc. This arrangement results in a steerable deflection of the steering end, wherein the steering end is not biased back into the rest position, since the opposing force is compensated by the torque produced by the energy storage element via the tension disc and the gear member. The deflected position of the steering end is maintained until the operator provides another input, e.g., moves the hull relative to the frame.

In one embodiment, teeth of the gear member mesh with a toothed rack fixed at the hull, wherein the toothed rack drives the gear member if the hull is displaced along the longitudinal hull axis relative to the frame. The toothed rack may be fixed at an inner surface of the hull. The gear member may comprise two toothed wheels, wherein one toothed wheel meshes with the toothed rack and the other toothed wheel is engaged with the elongated element, e.g., the toggle lever. Both toothed wheels may rotate about the same rotational axis and may provide a gear transmission provided by the diameter of the toothed wheels and/or the number of teeth of each toothed wheel in a known manner. A drive shaft of the gear member representing its rotational axis may be fixed to the frame. The gear transmission of the toothed wheels of the gear member supports a fine control of the steering end.

In one embodiment, the catheterization device comprises a friction mechanism comprising at least one friction pad fixed to the frame and at least one friction plate fixed to and moveable with the hull, wherein the surface of the friction pad is configured such that it rubs at the respective, opposite friction plate if the hull is displaced relative to the frame. The friction mechanism causes additional friction between the frame and the hull, such that the operator feels a resistance while displacing the hull relative to the frame. The additional friction provides a smooth operation and prevents unintentional deflections of the steering end since a certain force necessary to overcome the friction force is required to displace the hull.

In one embodiment, the friction mechanism comprises at least one pair of friction pads and a respective, opposite pair of friction plates, wherein one pair of friction pads are arranged at opposite ends of a compression spring biasing each friction surface of the friction pads against the respective opposite friction plate. The compression spring biases the friction pads against the friction plates with a constant and controlled force, resulting in a stick-slip free movement with constant friction allowing a sensitive displacement of the deflectable steering end. Further, the arrangement provides a permanent sliding contact between the hull and the frame, such that a potential canting is prevented.

In one embodiment the friction plate comprises at least one groove configured to indicate a defined position of the hull relative to the frame. The groove is, for example, arranged on a surface of the friction plate which is provided for the direct contact with the friction pad. In one embodiment, the at least one groove is positioned such that it indicates, for example, the rest position of the steering end as the defined position and/or a maximum deflection position of the steering end as the defined position. The operator may notice a small raise in friction if the position of the groove is crossed thereby receiving a haptic feedback that the defined position (e.g., the rest position or the maximum deflection position of the steering end) is reached. This gives the operator an information about the condition of the steering end in a situation where visible confirmation is absent.

In one embodiment, the at least one groove is arranged substantially perpendicular to the longitudinal hull axis because, in one embodiment, the hull axis is substantially the displacement direction of the hull relative to the frame. Orienting the groove perpendicular to this axis generates a sharp feedback, such that the operator feels directly when the operator reaches the respective defined position, resulting in a good tactile feedback.

In one embodiment the friction plate and/or the friction pad comprises at least one material of the group comprising PTFE and NBR shore gummy. Both materials are cost-effective and easy to process. Furthermore, this combination provides good sliding properties with little to no stick-slip effect with a constant friction.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawings, wherein

FIG. 1 depicts an embodiment of a catheterization device in a side view;

FIG. 2 depicts the catheterization device of FIG. 1, wherein its hull is transparent;

FIG. 3 depicts a side view of a control arrangement of the catheterization device of FIG. 1;

FIG. 4 depicts a top view of the control arrangement of FIG. 3;

FIG. 5 depicts the view of FIG. 4, wherein the steering end is in a rest position;

FIG. 6 depicts the view of FIG. 4, wherein the elongated element is within a pre-defined angular range corresponding to the rest position of the steering end;

FIG. 7 depicts the view of FIG. 4, wherein the elongated element is outside the predefined angular range by a small angular value;

FIG. 8 depicts the view of FIG. 4, wherein the elongated element is outside the predefined angular range by an angular value greater than the one shown in FIG. 7; and

FIG. 9 depicts a proximal end of the control arrangement of FIG. 3 and a friction mechanism in a perspective side view.

### DETAILED DESCRIPTION

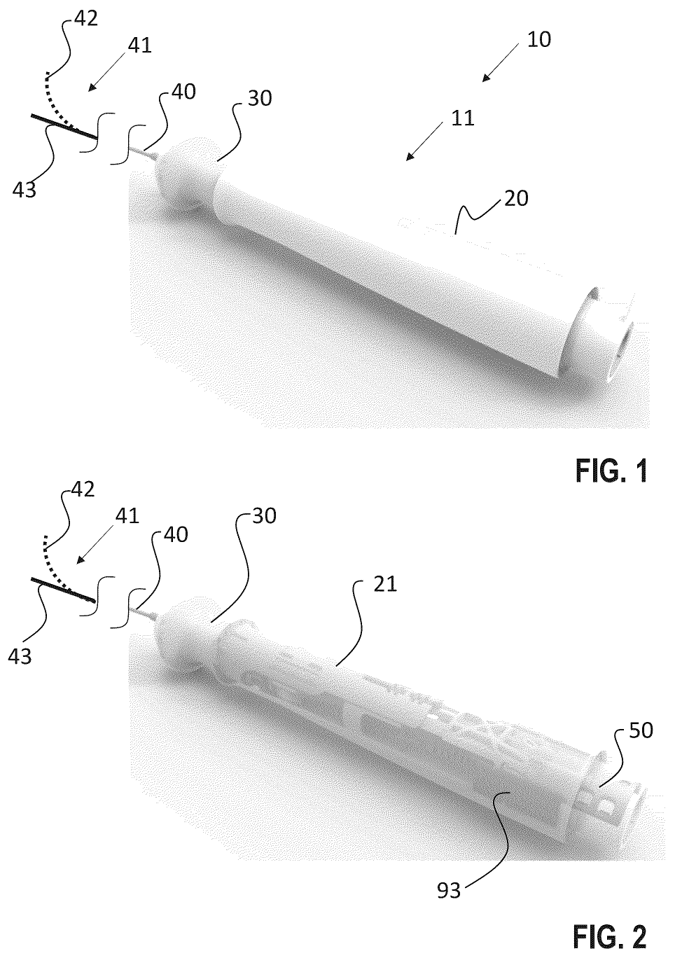

The embodiment of a catheterization device 10 as depicted in FIGS. 1 to 9 comprises a control device 11 with a hull 20, a frame 50 with a head 30, and a catheter 40 having a deflectable steering end 41, see FIG. 1. The deflectable steering end 41 can be deflected by displacing the hull 20 relatively to the frame 50 along the longitudinal axis of the hull, or by displacing the frame 50 relatively to the hull 20.

The head 30 forms a distal end of the frame, wherein the catheter 40 is fixed to the distal end of the head 30. In a deflected position the deflectable steering end 41 forms a curve 42, while in a rest position the deflectable steering end 41 forms substantially a straight line 43 (see FIGS. 1 and 2).

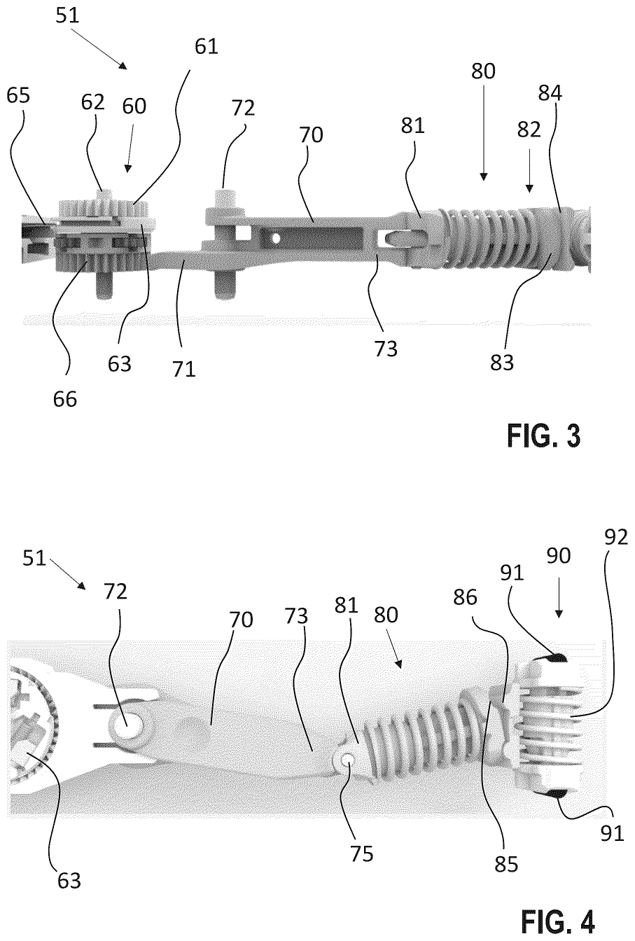

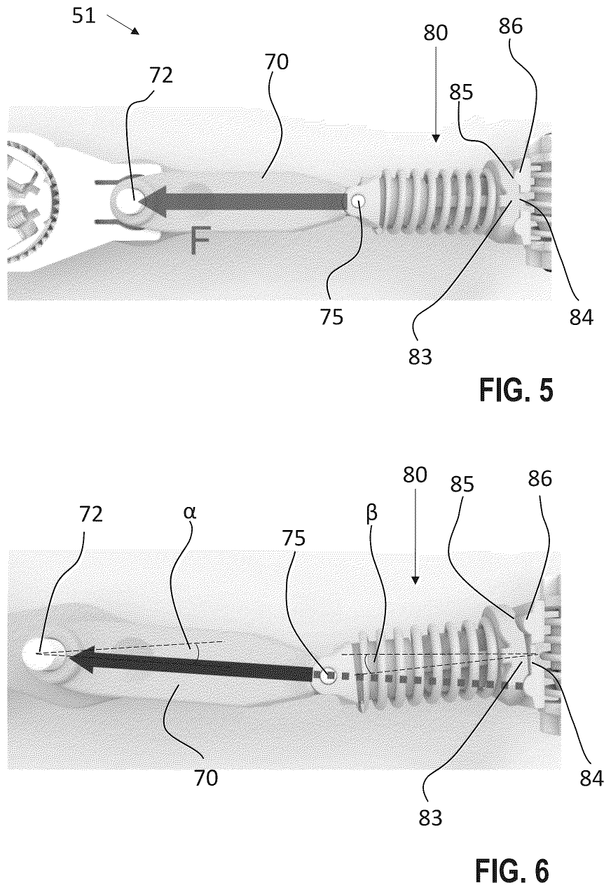

A toothed rack 21 is fixed to the hull 20. The toothed rack 21 meshes with a first gear wheel 61 of a gear member 60 as a part of a control arrangement 51 shown in FIGS. 3 and 4. The gear member 60 is rotatable fixed to the frame 50 via a shaft 62. The gear member 60 further comprises a tension disc 63 as a guide element. The tension disc 63 is connected in a movement-transmitting manner to the deflectable steering end 41 via two steering wires 65, see FIG. 3.

Displacing the hull 20 and thereby the toothed rack 21 relative to the frame 50 results in a rotation of the first gear wheel 61 and the tension disc 63, wherein the steering wires 65 transmit the movement of the tension disc 63 to the deflectable steering end 41, resulting in a deflection of the steering end 41.

In order to compensate opposing forces of the deflectable steering end 41, the gear member 60 comprises a second gear wheel 66 rotating with the first gear wheel 61 and the tension disc 63 and engaging a first end 71 of a toggle lever 70. The toggle lever 70 forms effectively the core of the control arrangement 51. The toggle lever 70 is rotatable about a first pivot axis 72, wherein the corresponding shaft is attached to the frame 70. A second end of the toggle lever 73 is attached to a first end 81 of an energy storage element 80 via a force transmitting element 75 forming a pin or alike, see FIG. 4. The energy storage element 80 may comprise, for example, a compression spring.

Thus, displacing the hull 20 relative to the displacement element 30 results in a pivoting movement of the toggle lever 70 about the first pivot axis 72 caused by the rotation of the first gear wheel 61 and the second gear wheel 62 meshing with the tooth at the first end 71 of the toggle lever 70.

A second end 82 of the energy storage element 80 comprises a generally planar or slightly curved first rolling surface 83 which interacts with a second rolling surface 84 attached to the frame 50 which is generally planar or slightly curved, as well. The first rolling surface 83 comprises two recesses 85 which are adapted to fit to a form, position and/or orientation of two ribs 86 formed on the second rolling face 84. The ribs 86 are aligned along a second pivot axis 87 and a third pivot axis 88 and arranged distanced and generally parallel to the first pivot axis 72. Further, the second pivot axis 87 is generally parallel to the third pivot axis 88. A cross section of each rib forms a segment of a circle which centers in the second or third pivot axis 87, 88. The plate-like element forming the second rolling surface 84 is fixed to the frame.

If the steering end 41 is in the rest position and the toggle lever 70 is in the corresponding rest position (see FIG. 5), the steering end 41 forms a straight line 43. In this position, the gear member axis 62, the first pivot axis 72 and the force transmitting element 75 form a line and the first rolling surface 83 and the second rolling surface 84 contact each other in a centered position, see FIG. 5. Thus, a compressive force F produced by the energy storage element 80 is applied straight into the force transmitting element 75 and the first pivot axis 72, such that no torque is applied to the gear member 60.

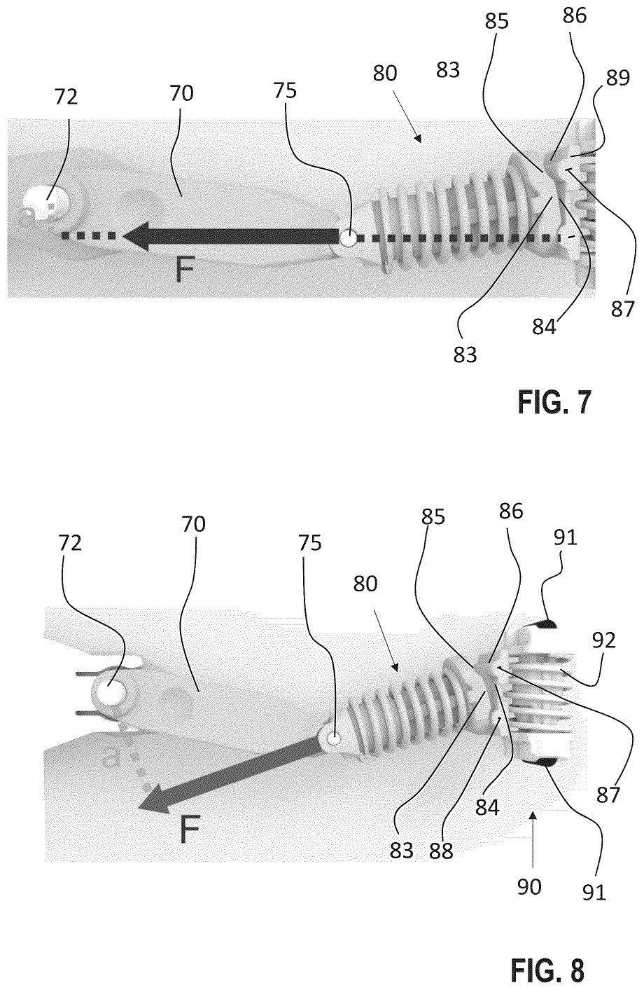

In a pre-defined angular range around the rest position of the steering end (see FIG. 6), displacing the hull 20 relative to the frame 50 does not result in a deflection of the deflectable steering end 41. However, such small displacement of the hull 20 results in a small pivoting movement of the toggle lever 70 within this pre-defined angular range (angle α) and a respective rotation of the energy storage element 80 (angle β in FIG. 6). Accordingly, the first rolling surface 83 of the energy storage element 80 rolls along the second rolling surface 84 between the second pivot axis 87 and the third pivot axis 88 (and back) like a rocking chair. Within this pre-defined angular range a the compressive force F points still into the direction of the first pivot axis 72 due to the rocking-chair movement (see FIG. 6), such that no torque component of the force is produced. Thereby unwanted jumping of the steering end tip caused by the slack of the steering wires 65 is prevented.

As soon as the relative displacement of hull 20 and frame 50 is greater, the rotation angle of the toggle lever 70 exceeds the pre-defined angular range, and the steering end 41 is deflected. In this state the energy storage element 80 provides a compensating force F and a compensating torque (see component a) to the toggle lever 70, which is transmitted to the gear member 60 assisting the operator in the deflection operation. With an increase of the pivot angle exceeding the pre-defined angular range of the toggle lever 70, the compensating force and the compensating torque increase as well, as shown if one compares FIG. 7 and FIG. 8. In this state the energy storage element 80 rotates either about the second pivot axis 87 or about the third pivot axis 88 (in the example shown in FIGS. 7 and 8 about the third pivot axis 88).

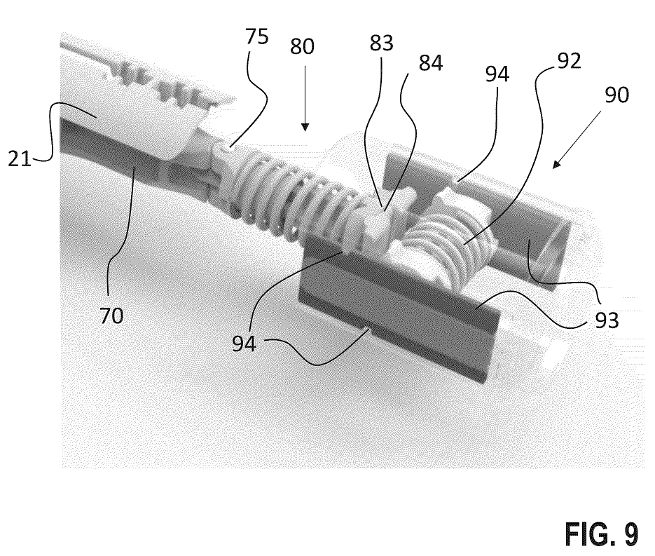

Proximal to the second rolling surface 84 a friction mechanism 90 is arranged having a pair of friction pads 91 and a compression spring 92. The compression spring 92 is guided by a respective recess of the frame 50 thereby allowing biasing the friction pads 91 by the compression spring 92 away from each other, perpendicular to the longitudinal axis of the hull 20 and against respective friction plates 93 fixed to the hull 20, as shown in FIG. 9. This means that each friction pad 91 interacts with one friction plate 93 providing a smooth operation of the catheterization device.

Each friction plate 93 comprise a groove 94 which indicates a defined position, for example, the rest position. Further, each groove 94 is arranged on a side of the friction plate 93, which contacts the corresponding friction pad 91. The material of the friction pad 91 may be, for example, NBR shore gummy and the surface of the friction plate 94, for example, PTFE.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCES

10 catheterization device
20 hull
21 toothed rack
30 head
40 Catheter
41 deflectable steering end
42 curve
43 straight line
50 frame
51 control arrangement
60 gear member
61 first gear element
62 gear member axis
63 tension disc
65 steering wire
66 second gear element
70 toggle lever
71 first end of the toggle lever
72 first pivot axis
72 second end of the toggle lever
75 force transmitting element
80 energy storage element
81 first end of the energy storage element
82 second end of the energy storage element
83 first rolling surface
84 second rolling surface
85 recess
86 rib
87 second pivot axis
88 third pivot axis
89 plate-like element
90 friction mechanism
91 friction pad
92 compression spring
93 friction plate
94 groove

1. A catheterization device comprising:
a catheter including a deflectable steering end having a rest position, and a control device for controlling the deflectable steering end, wherein the control device comprises a control arrangement having an elongated element which is mounted rotatably about a first pivot axis, wherein the control arrangement further comprises at least one guide element arranged at a distance from the first pivot axis, wherein at least one the guide element is rotatable with the elongated element about the first pivot axis or the guide element is rotatable mounted and coupled to the rotational movement of the elongated element, wherein the at least one guide element is connected in a movement-transmitting manner to the steering end,
wherein the control arrangement further comprises an energy storage element, which produces an auxiliary force assisting the rotation of the elongated element, wherein the elongated element has a force transmission element which is arranged at a distance from the first pivot axis and from the at least one guide element and which is coupled in a force-transmitting manner to a first end of the energy storage element,
wherein the energy storage element comprises a first rolling surface located at a second end of the energy storage element opposite to the first end and extending substantially parallel to the first pivot axis, wherein the first rolling surface is configured such that, if the elongated element rotates about the first pivot axis within a pre-defined angular range corresponding to the rest position of the steering end, the first rolling surface rolls along a second rolling surface thereby executing a combined translational and rotational movement along the second rolling surface between a second pivot axis and a third pivot axis, wherein the second pivot axis and the third pivot axis extend substantially parallel to the first pivot axis.

2. The catheterization device according to claim 1, wherein the second rolling surface comprises a first rib and a second rib extending substantially parallel to the first pivot axis, wherein the rolling movement of the first rolling surface along the first rib causes rotation of the second end of the energy storage element about the second pivot axis and wherein the rolling movement of the first rolling surface along the second rib causes rotation of the second end of the energy storage element about the third pivot axis if the elongated element rotates about the first pivot axis outside the pre-defined angular range.

3. The catheterization device according to claim 2, wherein the first rolling surface comprises a first recess and a second recess, wherein the first recess is adapted to be engaged with the first rib and the second recess is adapted to be engaged with the second rib.

4. The catheterization device according to claim 1, wherein the pre-defined angular range about which the elongated element rotates about the first pivot axis is an angular range between 10° and −10°, in particular between 7° and −7°, in particular between 4° and −4°.

5. The catheterization device according to claim 1, wherein the control device comprises a frame and a hull, which is displaceable relatively to the frame thereby causing rotation of the elongated element about the first pivot axis, wherein the displacement direction extends along a longitudinal hull axis, wherein the first pivot axis and the second rolling surface are fixed to the frame.

6. The catheterization device according to claim 1, wherein the elongated element is a toggle lever pivotable around the first pivot axis and the control arrangement further comprises a gear member, wherein a first end of the toggle lever engages with the gear member comprising the at least one guide element and a second end of the toggle lever is coupled to the force transmission element.

7. The catheterization device according to claim 6, wherein the toggle lever transmits a torque to a tension disc as the guide element arranged at the gear member to compensate the opposing force of the steering end of the catheter via the tension disc.

8. The catheterization device according to claim 6, wherein teeth of the gear member mesh with a toothed rack fixed at the hull, wherein the toothed rack drives the gear member if the hull is displaced along the longitudinal hull axis relative to the frame.

9. The catheterization device according to claim 5, further comprising a friction mechanism comprising at least one friction pad fixed to the frame and at least one friction plate fixed to and moveable with the hull, wherein the surface of the friction pad is configured such that it rubs at the respective, opposite friction plate if the hull is displaced relative to the frame.

10. The catheterization device according to claim 9, wherein the friction mechanism comprises at least one pair of friction pads and a respective, opposite pair of friction plates, wherein one pair of friction pads are arranged at opposite ends of a compression spring biasing each friction surface against the respective opposite friction plate.

11. The catheterization device according to claim 9, wherein the friction plate comprises at least one groove configured to indicate a defined position of the hull relative to the frame.

12. The catheterization device according to claim 11, wherein the at least one groove is arranged substantially perpendicular to the longitudinal hull axis.

13. The catheterization device according to claim 9, wherein the friction plate and/or the friction pad comprises at least one material of the group comprising PTFE and NBR shore gummy.
